(19) United States
(12) Patent Application Publication
(10) Pub. No.: US 2006/0263795 A1
(43) Pub. Date: Nov. 23, 2006

(54) Method and apparatus for facilitating the creation and analysis of microarrays

(75) Inventors: Jeffrey S. Williams, Saline, MI (US); David Byatt, Cambridgeshire (GB); David Crosby, Impington (GB); Jonathan I. Parker, Huntingdon (GB)

Correspondence Address: RADER, FISHMAN & GRAUER PLLC, 39533 WOODWARD AVENUE, SUITE 140, BLOOMFIELD HILLS, MI, 48304-0610, US

(73) Assignee: Genomic Solutions, Inc.

(21) Appl. No.: 11/320,051

(22) Filed: Dec. 28, 2005

Related U.S. Application Data
(63) Continuation of application No. 10/479,258.
(60) Provisional application No. 60/293,934, filed on May 29, 2001.

(51) Int. Cl. C12Q 1/68; C12M 1/34; B05D 3/02

(52) U.S. Cl. 435/6; 435/287.2; 427/2.11

(57) ABSTRACT

A substrate holder is provided to move a slide containing a microarray of biomaterial through a biomaterial analysis process. The substrate holder includes a substrate loader that allows loading of slides into the substrate holder with minimal contact to the slide, thereby minimizing the amount of contamination on the slide. The substrate holder can be used to transport a microarray of DNA through array fabrication, hybridization and analysis stations.

### FIELD OF THE INVENTION

The present invention relates generally to facilitating the creation and analysis of microarrays, and more particularly, the present invention relates to facilitating the creation and analysis of microarrays by using a substrate holder to minimize contamination of samples contained on a substrate.

### BACKGROUND OF THE INVENTION

The present invention described herein is used for material handling of substrates containing biological material for sampling (the “invention”). Many areas of scientific investigation require the use of biological material analysis. For example, in the field of genomics research, microarrays of known DNA base pair sequences are used to identify and detect expressed gene sequences in biological samples, or identify polymorphisms and mutations in the DNA of biological samples. Likewise, other biological material arrays such as modified and unmodified proteins and peptides, modified and unmodified nucleic acids, antibodies, antigens, carbohydrates, and other biopolymers are used to identify biological properties of like materials.

With respect to genomics research, microarrays of known DNA are prepared as a means to match known and unknown DNA samples based on hybridization principles, for example, to identify gene sequences or to determine gene expression levels. In one method, microarrays can be made by “spotting” collections of suspended, purified DNA strands onto a substrate. The substrate can be any device known to one skilled in the art for supporting biological material, such as DNA, as one example only. In a typical production method, the substrate, such as a glass slide, is loaded into a microarray production instrument and a microarray robot places drops or aliquots of individual DNA types onto the slide in a grid design. The grid may contain thousands of DNA spots of different base pair sequences (i.e. primary sequence) that are fixed to the substrate. The slide is then moved to a hybridization instrument or chamber where samples are probed for the presence and abundance of DNA or RNA (or mRNA) by hybridizing them to the prepared DNA microarray. If an individual cDNA probe in the sample is complimentary to the sequence of DNA on a given spot, the cDNA will hybridize to the spot, and the hybridization may be detected by its fluorescence. In this manner, each spot in the microarray may act to assay the presence of a different cDNA utilizing the sequence-specific affinity inherent to the formation of double-stranded nucleic acid polymers.

After the cDNA probes have been hybridized to the microarray and any free probes have been removed, the microarray is moved to a microarray scanner to be scanned to evaluate the comparative binding levels of individual probes. cDNA probes hybridized to DNA spots in the microarray can be detected through the use of different colored fluorophores or dyes that emit light at differential, characteristic wavelengths when excited by an illumination source in the microarray analyzer.

Microarray spots with more bound probe will fluoresce more intensely. The emitted light is captured by detector, such as a charge-coupled device (CCD) or a photo multiplier Tube (PMT), which records its intensity. The recorded data is stored or processed for further analysis.

To accomplish the above-described process, the slides containing the microarray and DNA test probes must be moved through all the different stations as described above. These stations include the microarrayer where the microarray is fabricated onto the slide, the hybridization station where the cDNA is hybridized to the microarray, and the washing and drying station where excess spotted cDNA, which has not hybridized to the microarray, is washed from the slide. The slides must also be moved from the drying station to the microarray analyzer. From the microarray analyzer, the slides must be moved to a storage or disposal facility.

Conventional processing includes manual handling of the slides for movement among all the above described stations. In addition to being cumbersome and labor intensive, manual handling of slides can contaminate the slides, introduce technical error and distort the overall results of the study. Specifically, conventional slides have little auto-fluorescence to reduce background illumination. This helps ensure that the only illumination generated in the microarray analyzer comes from the hybridized DNA and cDNA, or other biomolecule under investigation. The remaining illumination is preferably reduced and filtered out. This ensures that the illumination wavelengths and intensity levels recorded by the microarray analyzer are from the hybridized DNA and cDNA.

Contamination by fingerprints or other debris caused by material handling adds to the auto-fluorescence of the slides, thereby increasing the background light and distorting the results of the biomaterial testing. The present invention was developed in light of these and other drawbacks.

### SUMMARY OF THE INVENTION

To address these and other drawbacks, the present invention provides a substrate holder that allows a substrate or any other surface amenable to fluidic-based hybridization, to be transported through a biomaterial analysis with minimal handling of the substrate.

In one aspect of the present invention, a method of analyzing biomaterial includes a series of steps. At least one substrate is loaded into a substrate holder. Next, a microarray of biomaterial is deposited on the substrate in the substrate holder. The substrate holder is then moved to a hybridization station where a second biomaterial is hybridized to the biomaterial in the microarray. The substrate holder is then moved to a microarray analyzer station where the hybridized material is analyzed.

In another aspect, a substrate holder is provided that includes a support structure having a substrate aperture recessed into a face of the support structure. The substrate aperture is sized to accommodate a substrate. A plurality of locking devices is disposed around the periphery of the substrate aperture to lock the substrate into place.

In another aspect of the present invention, a substrate loading kit is provided including a plurality of components. The substrate holder is provided having a substrate aperture for receiving a substrate. The substrate holder has a plurality of locking devices disposed around the periphery of the substrate aperture for locking the substrate into position. A substrate loader is provided that has members adapted to engage and open locking devices when the substrate holder is moved with respect to the substrate loader.

In another aspect of the present invention, a method for loading substrates into the substrate holder is provided. The substrate holder has a substrate aperture for accommodating a substrate. A plurality of spring apertures is disposed around the periphery of the substrate aperture. Each spring aperture contains a respective spring that extends through the aperture and into the substrate aperture. The substrate loader includes a base portion and a plurality of pins extending from the base portion. The substrate holder is placed on the substrate loader such that each of the pins extends through a respective one of the spring apertures. The substrate holder is then moved with respect to the substrate loader such that each of the pins abuts a respective one of the springs to thereby move the springs out of the substrate aperture. The substrate is then placed into the substrate aperture and the substrate holder is removed from the substrate loader. Once the substrate holder is removed from the substrate loader, the springs clamp against edge portions of the substrate to hold it into place.

Other objects and features of the present invention will become more readily apparent from a better understanding of the preferred embodiments described below with reference to the following drawings.

### BRIEF DESCRIPTION OF THE DRAWINGS

The features and inventive aspects of the present invention will become more apparent upon reading the following detailed description, claims and drawings, of which the following is a brief description:

FIG. 1 is a perspective view of a substrate holder according to the present invention;

FIG. 2 is a perspective exploded view of II in FIG. 1;

FIG. 3 is a perspective exploded view of III in FIG. 1;

FIG. 4 is a perspective view of a substrate loader for a substrate holder according to the present invention;

FIG. 5A is a plan operational view of a substrate holder being used in conjunction with a substrate loader according to the present invention;

FIG. 5B is a plan operational view of a substrate holder being used in conjunction with a substrate loader according to the present invention;

FIG. 6 is a perspective view of stacked substrate holders according to the present invention; and

FIG. 7 is a flow chart depicting the operation of a substrate holder being used in conjunction with a biological material study according to the present invention.

### DETAILED DESCRIPTION OF THE INVENTION

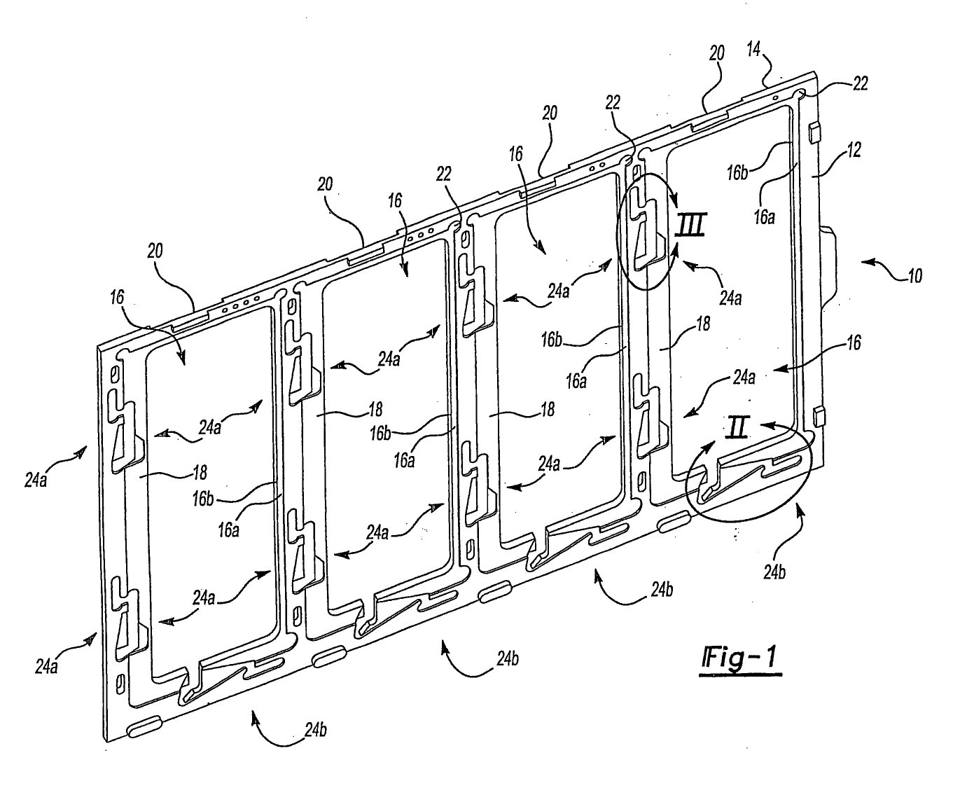

Referring now to FIG. 1, a substrate holder 10 according to the present invention is shown and described. The substrate holder 10 has a first side 12 oppositely disposed from a second site 14. A plurality of apertures 16, pass from the first side 12 to the second side 14. Each aperture 16 has a lengthwise edge and a widthwise edge. The lengthwise edge is longer than the widthwise edge to accommodate a slide or other similarly sized substrate, as will be described. Although the preferred embodiment shown in FIG. 1 includes four separate apertures, it is understood that other configurations of substrate holder 10 can be provided including only one aperture 16, two or three apertures 16, or more than four apertures 16 as shown.

Step 18 extends around the periphery of each aperture 16 and is recessed from first side 12. Step 18 is preferably recessed from first side 12 to a depth that allows a substrate such as a slide, as one example only, to sit on step 18 while being about flush with the surface of first side 12. Inner walls of step 18 define a smaller aperture 16B, while the portion of aperture 16 meeting first side 12 defines larger aperture 16A.

A match corner 22 is provided at a corner area of the larger aperture 16A. Match corner 22 opens into a radius to ensure that a corner of a slide resting in aperture 16 does not abut the structure of substrate holder 10. Additionally, match corner 22 acts as a reference point for orienting the substrate holder during processing and orienting the slides in the substrate holder.

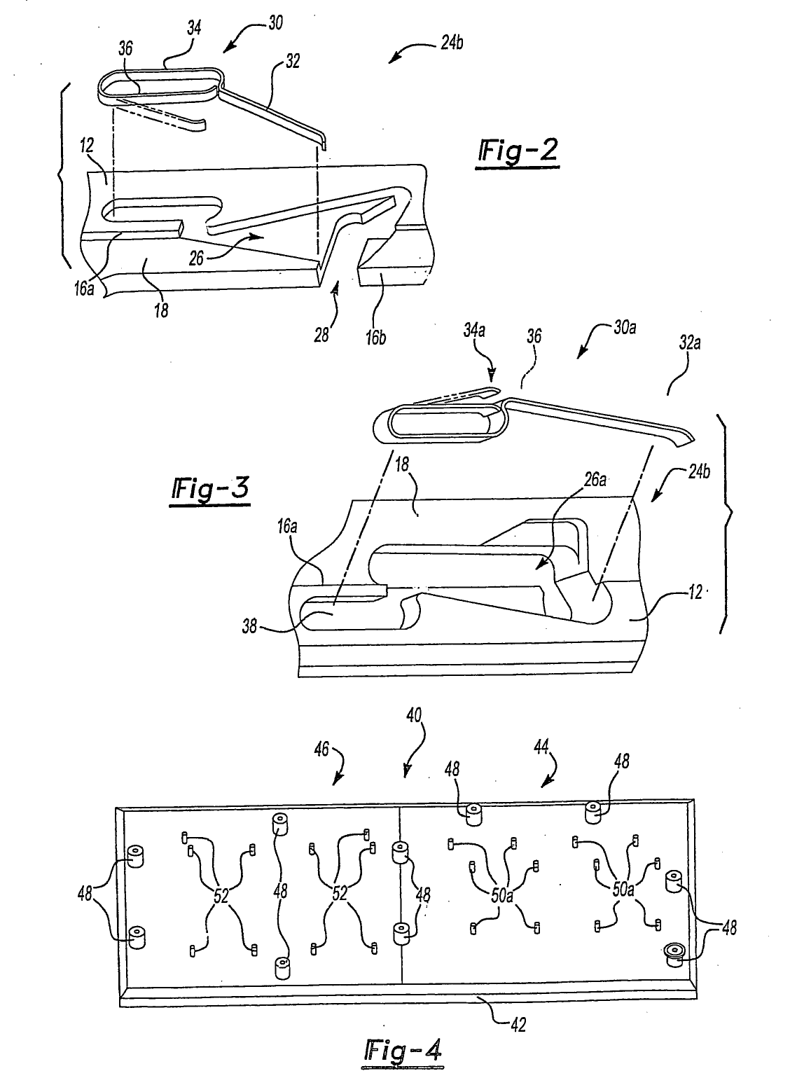

Spring loaders 24A are disposed along the lengthwise portion of larger aperture 16A. Likewise, spring loaders 24B are disposed along the widthwise portion of larger aperture 16A. Referring to FIG. 2, a magnified exploded view of spring loader 24B is shown and described. Spring loader 24B includes a spring aperture 26 which bridges across the boundary of larger aperture 16A such that one portion of spring aperture 26 recesses from first side 12 and a second portion of spring aperture 26 recesses from step 18. Groove 28 is a portion of aperture 26 that extends from the boundary of smaller aperture 16B to accommodate a pin from a loading mechanism. As such, groove 28 passes through the entirety of substrate holder 10, while the remainder of aperture 26 is recessed to a predetermined depth to accommodate spring 30.

Spring 30 includes substrate holder portion 32 and seating portion 34. A latch 36 is provided with spring 30 that is biased toward an open position. To assemble, the latch is moved to a closed position, and spring 30 is positioned into aperture 26. When released, latch 36 biases against inside walls of spring aperture 26 to grip thereagainst for support. Preferably, the portion of spring aperture 26, not including groove 28, is recessed to a depth that allows spring 30 to sit therein without projecting above the face of first side 12.

Referring now to FIG. 3, a magnified exploded view of spring loader 24A is shown and described. Like spring loader 24B, spring loader 24A includes a spring aperture 26A that bridges across the outer wall defining larger aperture 16A. Accordingly, one portion of spring aperture 26A recesses from first side 12 while a second portion of spring aperture 26A recesses from step 18. A spring step 38 is recessed to a depth from first side 12 sufficient to allow spring 30A to be seated therein while remaining flush with the top surface of first side 12. The remainder of spring aperture 26A passes through substrate holder 10 in its entirety to allow for a pin of a substrate holder to pass therethrough as will be described in greater detail. Like spring 30, spring 30A has a substrate holder portion 32A and a seating portion 34A. Seating portion 34A includes a latch 36, which is also biased towards an open position to grip walls of spring aperture 26A that recess from first side 12.

Referring now to FIG. 4, a substrate loader 40 is shown and described. Substrate loader 40 includes a rectangular based portion 42 divided into a loading side 44 and an unloading side 46. A plurality of support posts 48 extend from the surface of base portion 42. Likewise, a plurality of spring loader pins 50 and spring loader pins 50A extend from the loading side 44 of base portion 42. Unloading pins 52 extend from unloading side 46 of base portion 42. It is noted however, that substrate loader 40 can be any other configuration besides rectangular and that loading side 44 and unloading side 46 can be separated.

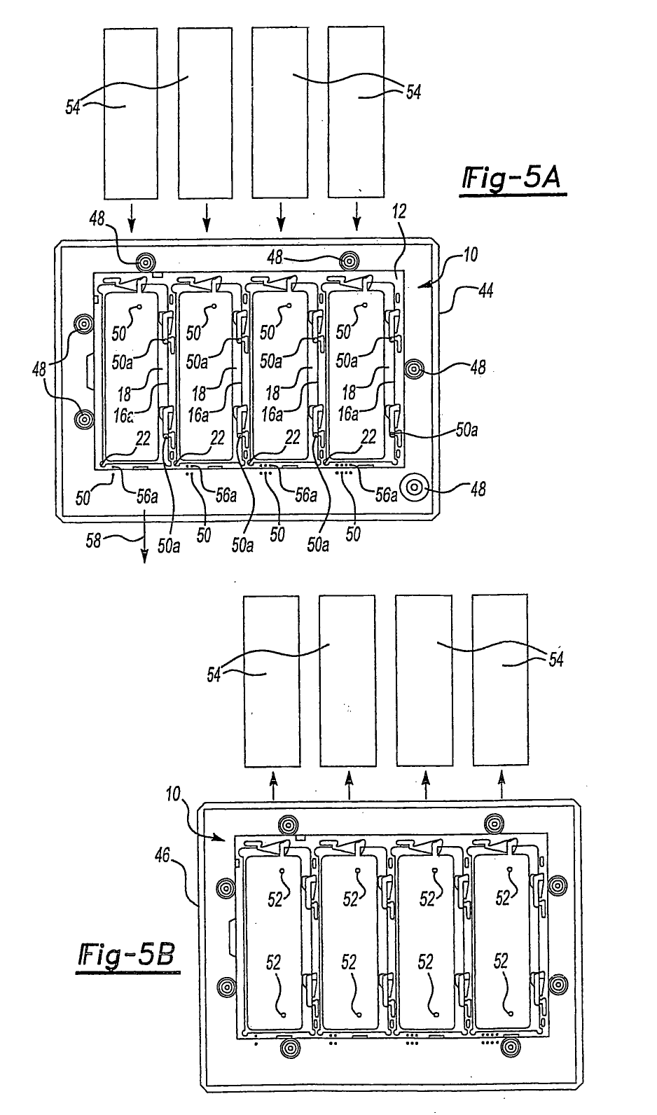

Referring now to FIGS. 5A and 5B, the operation of loading and unloading substrate holder 10 is described. For ease of explanation, loading side 44 and unloading side 46 are shown as separate entities. However, it is understood that loading side 44 and unloading side 46 may be joined or may be separate, as shown respectively in FIG. 4 or FIG. 5. In FIG. 5A, a plurality of slides 54 are to be loaded into substrate holder 10. Substrate holder 10 is dropped onto loading side 44 such that spring loader pins 50 sit below pin groove 28 and pins 50A sit in a bottom portion of spring aperture 26A (with respect to the orientation of the figure). Substrate holder 10 is oriented such that markings 56 on loading side 44 correspond to markings 56A on substrate holder 10. This helps act as an orientating device for determining the orientation of the substrate holder and corresponding slides. Also, support posts 48 provide additional locating for substrate holder 10.

Next, substrate holder 10 is pulled in the direction of arrow 58 which causes pins 50A to flex substrate holder portion 32A of spring 30A in a direction toward the outer periphery of substrate holder 10. Likewise, movement of substrate holder 10 in the direction of arrow 58 causes spring loader pins 50 to move substrate holder portion 32 in a direction toward the outer periphery of substrate holder 10. This moves substrate holder portions 32 and 32A of respective springs 30 and 30A out of the way of larger aperture 16A to allow slides 54 to be loaded therein. Slides 54 are dropped into larger apertures 16A of substrate holder 10. The face of step 18 is recessed from first side 12 to allow slides 54 to sit approximately flush with the surface of first side 12. As is noted, step 18 extends around the entire periphery of larger aperture 16A such that each slide 54 is supported around its periphery by step 18.

Once the slides are positioned in larger aperture 16A, substrate holder 10 is removed from loading side 44. This removal causes spring loader pins 50 and 50A to move out of contact with substrate holder portions 32 and 32A. As such, substrate holder portions 32 and 32A bias against the outer periphery of slide 54 to thereby grip and drive slides 54 toward match corner 22.

Referring now to FIG. 5B, unloading of slides 54 is accomplished by dropping substrate holder 10 onto unloading side 46. Here, unloading pins 52 extend upward a greater distance than the bottom of each slide 54. Accordingly, when substrate holder 10 is dropped onto unloading side 46, pins 52 push slides 54 out of larger aperture 16A, thereby releasing it from the grip of springs 30 and 30A.

It is noted that other configurations of the substrate holder 10 may be used. Such configurations need not include the specific locking or support mechanisms disclosed herein and merely need to provide a support structure that allows slide handling without contamination. Moreover, although substrate holder 10 is shown accommodating four slides, other variations of the number of slides accommodated may be used. Specifically, substrate holder 10 may accommodate only one slide or may accommodate a large number of slides and the present invention is not limited to that disclosed herein.

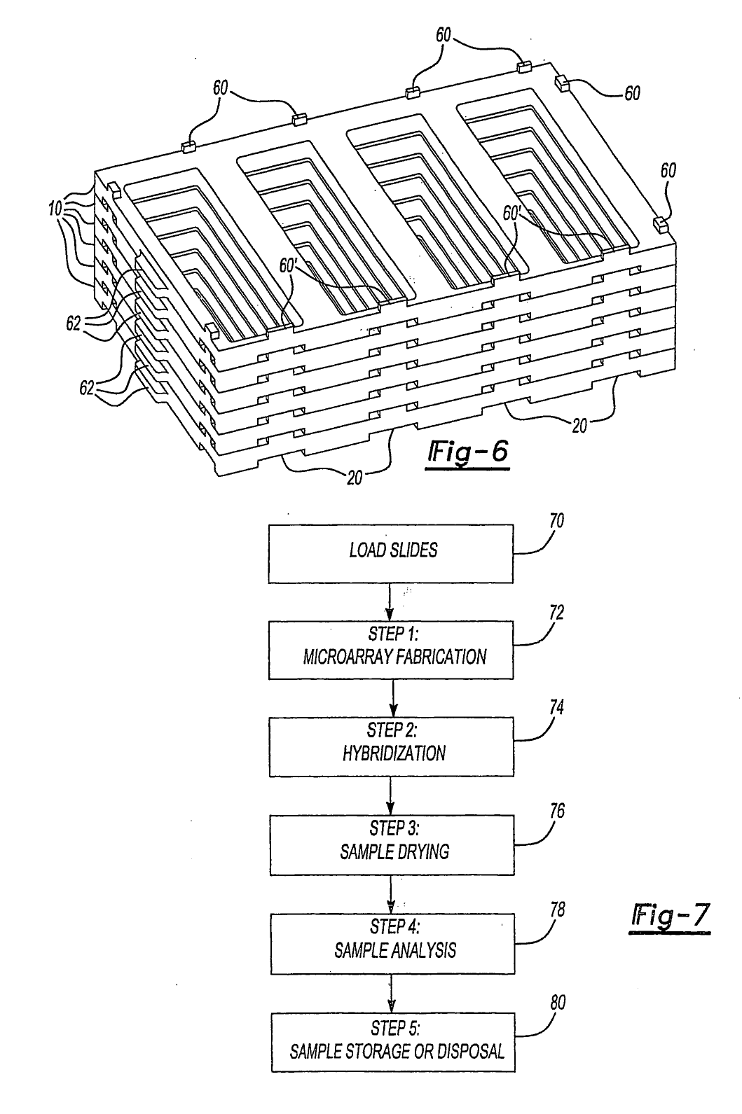

Referring now to FIG. 6, a plurality of stacked substrate holders 10 is shown. Such stacking allows ease of material handling as well as the application of automation such as robotics and robotic arms to move a substrate holder from the stack and into a machine for processing. To accommodate this stacking technique, each substrate holder 10 includes indentations 20 on second side 14 and extensions 60 that extend from the first side 12. The location of indentations 20 correspond with the location of extension 60 such that extensions 60 on lower stack level substrate holders reside within indentations 20 of upper stack level substrate holders. This allows the stack to be configured in a semi-locking fashion. Additionally, lips 62 are provided at edge portions of each substrate holder 10 such that the substrate holders 10 can be easily separated.

Referring now to FIG. 7, the operation of the present invention in conjunction with testing of biological samples is described. In step 70, the substrate holder 10 is loaded with slides. As described above, the substrate holder 10 according to the present invention may be adapted to accommodate any number of slides including one slide or multiple slides. Preferably, substrate holder 10 in the described preferred embodiment accommodates four separate slides.

In step 72, the biomaterial, which is to be tested, is deposited on each slide as a microarray. The biomaterial may be protein to conduct a protein study, carbohydrate material to conduct a carbohydrate study, DNA strands to conduct a DNA study or any other biological material for a similar study. By way of non-limiting example, the microarray in step 72 includes strands of DNA used to conduct a DNA study for genomic research.

The microarrays in step 72 are fabricated by “spotting” collections of suspended, purified DNA strands onto the slide contained in the substrate holder. A microarray robot places drops of individual DNA types onto the slide. Each slide is spotted with thousands of DNA spots of different base pair sequences that affix to the slide. To accomplish the microarray fabrication, the substrate holder is loaded into a microarrayer to allow the microarray robot to conduct spotting. Multiple substrate holders 10 can be loaded into the microarrayer to allow the microarray robot to spot numerous slides at one time. Or, few substrate holders can be loaded into the microarrayer. Match corner 22 provides a reference from which to orient the substrate holder 10 throughout the entire DNA study process.

Once the microarray is fabricated, the substrate holder 10 is transported to a hybridization station in step 74. The method of transport is preferably accomplished by gripping the substrate holder 10, not the actual slides themselves. This limits the amount of contamination on the slides. The transport may be accomplished by use of human hands or robotics.

The substrate holders 10 are loaded into a hybridization machine to conduct hybridization. Here, a cDNA probe is washed over the spotted slide and cooked under a specific temperature and mixing conditions. If the individual cDNA probe is complimentary to the sequence of the DNA on a given spot, the cDNA will hybridize to the spot. The cDNA from each and any given probe is treated with colored fluorophores or dyes that emit light at a differential, characteristic wavelength excited by an illumination source such as a microarray analyzer.

In step 76, non-hybridized DNA material is washed clean from the slide and the remaining hybridized material is dried. To accomplish this, the slide is transported from the hybridization station in step 74 to a washing and drying station in step 76. Again, the slide is preferably transported by handling the substrate holder, not the slides themselves. This process may also be accomplished by automation or robotics or by human hands. As the method of transportation need not grip the slides, contamination is reduced.

In step 78, the substrate holder 10 is loaded into a microarray scanner. The cDNA probes have been treated with colored fluorophores or dyes that emit that differential, characteristic wavelength when excited by an illumination source. As such, the microarray scanner emits the characteristic wavelengths to illuminate the fluorophores. The illumination of the microarray spots is captured by detector, such as a charged-coupled device (CCD) or a photo-multiplier tube (PMT), which records the intensity of the illuminated spots. The recorded data is stored or processed for further analysis.

A portion of the substrate holder 10, not including the slides 54, is once again handled to transport the substrate holder 10 from the washing and drying station in step 76 to the microarray analyzer in step 78. The orientation of the slides is maintained in the microarray analyzer by using the match corner 22 as a reference point for the slides. Moreover, slide loading into the microarray analyzer in step 78 can be conducted by automation. This automation is facilitated by the stacking ability described above. Specifically, the substrate holders 10 can be stacked next to a robotic arm or other automated machine and loaded into an analyzer such as the microarray analyzer.

In step 80, the slides are removed from the substrate holder according to the discussion set out above. The slides are then stored or disposed and the substrate holder 10 is recirculated for reloading in step 70.

It will be understood by one skilled in the art that the present invention may be used in conjunction with other substrates in addition to slides, which include any medium or device for supporting an array of biological material understood to one skilled in the art, and the present invention is in no way limited to the description disclosed herein.

Preferred embodiments of the present Invention have been disclosed. A person of ordinary skill in the art would realize, however, that certain modifications would come within the teachings of this Invention, and the following claims should be studied to determine the true scope and content of the invention. In addition, the methods and structures of the present invention can be incorporated in the form of a variety of embodiments, only a few of which are described herein. It will be apparent to the artisan that other embodiments exist that does not depart from the spirit of the invention. Thus, the described embodiments are illustrative and should not be construed as restrictive.

What is claimed is:

1. A method of conducting a study of biomaterial comprising the steps of:
loading at least one substrate into a substrate holder;
moving the substrate holder and the at least one substrate to a microarrayer;
depositing a microarray of a first biomaterial on the at least one substrate in the substrate holder at the microarrayer;
moving the substrate holder and at least one substrate to a hybridization station;
hybridizing a second biomaterial to the first biomaterial on at least one substrate in the substrate holder at the hybridization station;
moving the substrate holder and at least one substrate to a substrate dryer;
drying the biomaterial on at least one substrate hybridized in the previous step;
moving the substrate holder and the at least one substrate to a microarray analyzer station; and
analyzing the second biomaterial on the at least one substrate in the substrate holder at the microarray analyzer.

2. The method according to claim 1, wherein the biomaterial is a plurality of polynucleotides.

3. The method according to claim 1, wherein the second biomaterial is cDNA probes.

4. The method according to claim 1, wherein the second biomaterial is oligonucleotide probes.

5. The method according to claim 1, wherein the step of loading the at least one substrate further includes:
opening a substrate lock on the substrate holder with a substrate loader;
loading the at least one substrate into the substrate loader; and
closing the lock with the substrate loader to lock the at least one substrate in the substrate holder.

6. The method according to claim 5, wherein:
the substrate loader includes at least one substrate aperture for accommodating the at least one substrate; and
the substrate lock includes a plurality of spring loaders disposed around a periphery of the substrate aperture for locking and unlocking the substrate in the substrate holder.

7. The method according to claim 6, wherein the step of loading the at least one substrate further comprises:
moving the substrate holder with respect to the substrate loader to abut each of a plurality of pins extending from the substrate loader against a respective one of the spring loaders to open the substrate aperture and allow the substrate to be loaded in the substrate holder; and
removing the substrate holder from the substrate loader to move each of the pins from abutment against respective ones of the spring loaders to allow the spring loaders to lock against an outer edge of the substrate.

8. The method according to claim 1, further comprising the step of removing the at least one substrate from the substrate holder after the step of analyzing the second biomaterial in the microarrayer.

9. The method according to claim 8, wherein the step of removing the at least one substrate further comprises:
pushing the at least one substrate with pins extending from a substrate loader to disengage the at least one substrate from a substrate locking mechanism.

10. The method according to claim 1, wherein the step of moving the substrate holder and the at least one substrate to the microarray analyzer station further comprises the steps of:
stacking the substrate holder on a plurality of other substrate holders; and
moving the substrate holder from the stack of other substrate holders to the microarray analyzer station.

11. The method according to claim 10, wherein the step of moving a substrate holder from the plurality of other substrate holders to the microarray analyzer station is accomplished by using a robotic device.

12. The method according to claim 1, wherein the steps of moving the substrate holder to the hybridization station and moving the substrate holder to the microarray analyzer station is accomplished without contacting the substrate.

13. The method according to claim 1, wherein the substrate holder accommodates a plurality of substrates.

14. The method according to claim 1, further comprising the steps of:
moving the substrate holder and the at least one substrate to a slide dryer after the step of hybridizing; and
drying the at least one substrate in the dryer before the step of moving the substrate holder and the at least one substrate to the microarray analyzer station.

15. The method according to claim 1, wherein the step of depositing the microarray is performed by a microarraying robot.

16. A substrate holder comprising:
a support structure having at least one substrate aperture recessed into a face of the support structure, the aperture sized to accommodate a substrate; and
a plurality of substrate locking devices disposed around a periphery of the substrate aperture to lock the substrate in the aperture of the support structure.

17. The substrate holder according to claim 16, wherein the support structure further comprises:
a plurality of substrate apertures recessed from the face of the support structure, each of the plurality of substrate apertures sized to accommodate a respective one of a plurality of substrates; and
each of the plurality of substrate apertures having respective pluralities of substrate locking devices to lock each of the plurality of substrates to respective ones of the plurality of apertures in the support structure.

18. A substrate loading kit comprising:
a substrate holder having at least one substrate aperture recessed from a face of the substrate holder for receiving a substrate, the substrate holder having a locking device disposed around a periphery of the substrate aperture; and
a substrate loader having a member adapted to engage the locking device to open or close the locking device when the substrate holder is moved with respect to the substrate loader.

19. The substrate loading kit according to claim 18, wherein:
the member is a plurality of pins extending from the substrate loader;
the locking device is a plurality of locking devices, each of the plurality of locking devices includes:
a spring aperture; and
a spring mounted within the spring aperture and extending through the spring aperture and into the substrate aperture;
wherein movement of the substrate holder with respect to the substrate loader causes each one of the plurality of pins to abut each spring of the plurality of locking mechanisms to move each spring out of the substrate aperture.

20. The kit according to claim 18, further comprising a second set of pins extending from the substrate loader adapted to push the substrate out of the substrate aperture when the substrate abuts the second set of pins.

21. A method for loading substrates into a substrate holder comprising the steps of:
providing a substrate holder having:
at least one substrate aperture for accommodating a substrate;
a spring aperture disposed around a periphery of the substrate aperture;
a spring positioned in the spring aperture such that a portion of the spring extends into the substrate aperture;
providing a substrate loader having:
a base portion;
a pin extending from the base portion;
placing the substrate holder on the substrate loader such that the pin extends through the spring aperture;
moving the substrate holder with respect to the substrate loader to abut the spring with the pins to move the spring out of the substrate aperture;
placing the substrate into the substrate aperture; and
removing the substrate holder from the substrate loader to cause the spring to clamp against an edge portion of the substrate.

22. A method of conducting a study of biomaterial comprising the steps of:
loading at least one substrate into a substrate holder, the substrate having a preprinted microarray of a first biomaterial;
moving the substrate holder and the at least one substrate to a hybridization station;
hybridizing a second biomaterial to the first biomaterial on the at least one substrate in the substrate holder at the hybridization station;
moving the substrate holder and the at least one substrate to a microarray analyzer station; and
analyzing the second biomaterial on the at least one substrate in the substrate holder at the microarray analyzer.

23. The method according to claim 22, further comprising the steps of:
moving the substrate holder and the at least one substrate to a slide dryer after the step of hybridizing; and
drying the at least one substrate in the dryer before the step of moving the substrate holder and the at least one substrate to the microarray analyzer station.
